(19) United States
(12) Patent Application Publication
(10) Pub. No.: US 2020/0315462 A1
(43) Pub. Date: Oct. 8, 2020

(54) INFORMATION PROCESSING APPARATUS, INFORMATION PROCESSING METHOD, AND INFORMATION PROCESSING PROGRAM

(71) Applicant: OMRON HEALTHCARE CO., LTD., Muko-shi (JP)

(72) Inventors: Naoki TSUCHIYA, Tokyo (JP); Yoshiyuki MORITA, Nagaokakyo-shi (JP); Yasushi MATSUOKA, Kyoto (JP); Toru DENO, Kyoto (JP); Kosuke INOUE, Kyoto (JP)

(73) Assignee: OMRON HEALTHCARE CO., LTD., Muko-shi (JP)

(21) Appl. No.: 16/910,232

(22) Filed: Jun. 24, 2020

Related U.S. Application Data
(63) Continuation of application No. PCT/JP2018/046247, filed on Dec. 17, 2018.

(30) Foreign Application Priority Data
Dec. 27, 2017 (JP) 2017-252505

(51) Int. Cl. A61B 5/022; A61B 5/00; A61B 5/024

(52) U.S. Cl. CPC A61B 5/022; A61B 5/742; A61B 5/681; A61B 2562/0219; A61B 5/02444; A61B 2562/0247; A61B 2562/0271; A61B 5/02438

(57) ABSTRACT

An apparatus according to an aspect of the present invention includes an acquisition unit is configured to acquire a blood pressure value of a subject measured by a blood pressure measurement unit, a first pulse rate of the subject at a normal time, and a second pulse rate of the subject in a time period in which the blood pressure value is measured, a calculation unit is configured to calculate a first tension degree of an autonomic nerve of the subject based on the first pulse rate and to calculate a second tension degree of the autonomic nerve of the subject based on the second pulse rate, and a determination unit is configured to determine a type of blood pressure of the blood pressure value based on the blood pressure value, the first tension degree, and the second tension degree.

### CROSS-REFERENCE TO RELATED APPLICATIONS

This application is a Continuation Application of PCT Application No. PCT/JP2018/046247, filed Dec. 17, 2018 and based upon and claiming the benefit of priority from Japanese Patent Application No. 2017-252505, filed Dec. 27, 2017, the entire contents of all of which are incorporated herein by reference.

### FIELD

The present invention relates to an information processing apparatus, an information processing method, and an information processing program for processing a measured blood pressure value.

### BACKGROUND

The blood pressure monitor includes a portable type device that measures blood pressure by wrapping a cuff around an arm or the like, and a stationary type device that measures blood pressure by inserting an arm into a measurement unit in which a cuff is built. In recent years, wearable blood pressure monitors have been developed. For example, as one of them, there is known a tonometry type blood pressure measurement device capable of measuring vital information such as a pulse rate and a blood pressure value using information detected by a pressure sensor in a state where the pressure sensor is in direct contact with a biological site through which an artery such as a radial artery of a wrist passes (for example, see Jpn. Pat. Appin. KOKAI Publication No. 2017-006672). Furthermore, as other types of wearable blood pressure monitors, a blood pressure monitor using an oscillometric method, a trigger blood pressure monitor that estimates blood pressure fluctuation by a pulse transit time (PTT) method and measures a blood pressure value using the fluctuation as a trigger, and the like are also known.

### SUMMARY

However, no matter what type of blood pressure monitor is used, only the blood pressure value can be obtained, and the type of hypertension cannot be determined. It is known that hypertension includes, for example, persistent hypertension in which the blood pressure value is steadily higher than the normal value, and stress-induced hypertension in which the blood pressure value rises due to stress, and stress-induced hypertension includes white coat hypertension in which the blood pressure value rises due to stress and tensing caused by seeing the white coats of doctors, nurses, and the like, and workplace hypertension in which the blood pressure value rises due to excessive demands in the workplace or stress caused by interpersonal relationships. Accurate determination of the type of hypertension is extremely important in treating hypertension.

An information processing apparatus according to a first aspect of the present invention includes a blood pressure value acquisition unit configured to acquire a blood pressure value of a subject measured by a blood pressure measurement unit, a pulse rate acquisition unit configured to acquire a first pulse rate of the subject at a normal time and a second pulse rate of the subject in a time period in which the blood pressure value is measured, a calculation unit configured to calculate a first tension degree of an autonomic nerve of the subject based on the first pulse rate and to calculate a second tension degree of the autonomic nerve of the subject based on the second pulse rate, and a determination unit configured to determine a type of blood pressure of the blood pressure value based on the blood pressure value, the first tension degree, and the second tension degree.

According to the first aspect, the stress at the time of measuring the blood pressure value of the subject is determined based on the pulse rate at the time of measuring the blood pressure value of the subject and the pulse rate at a normal time of the subject. Then, based on the measured blood pressure value and the determination result of the stress, it is determined whether or not the blood pressure value is hypertension, and if the blood pressure value is hypertension, the type thereof is determined. Therefore, in addition to whether or not the measured blood pressure value corresponds to hypertension, it is possible to determine whether or not the hypertension is caused by the tension of the autonomic nerve.

According to the guidelines for the management of hypertension in Japan, a blood pressure value at a normal time such as home blood pressure is indispensable for determining the type of hypertension. However, according to the first aspect of the present invention, it is possible to determine the type of hypertension even for a person whose blood pressure value cannot be acquired at a normal time, such as a person who does not have a habit of measuring blood pressure at home or the like or a patient who neglects blood pressure measurement.

In an information processing apparatus according to a second aspect of the present invention, the determination unit is configured to determine whether or not the blood pressure value is classified as hypertension based on the blood pressure value, and when it is determined that the blood pressure value is classified as hypertension, the determination unit is configured to determine a type of the classified hypertension based on the first tension degree and the second tension degree.

According to the second aspect, the process of determining the type of hypertension is performed only when the measured blood pressure value is classified as hypertension. Therefore, when the measured blood pressure value is not classified as hypertension, the process of determining the type of hypertension is omitted, and the processing load is reduced accordingly.

An information processing apparatus according to a third aspect of the present invention further includes a determination unit configured to determine whether or not the subject is in a stressed state at the time of measuring the blood pressure value, based on the first tension degree and the second tension degree. When it is determined that the subject is in the stressed state at the time of measuring the blood pressure value, the determination unit is configured to determine that the blood pressure value is suspected of being stress-induced hypertension.

According to the third aspect, when the measured blood pressure value is classified as hypertension, it can be determined that the type of hypertension is suspected of being stress-induced hypertension defined in the guidelines for the management of hypertension in Japan.

In an information processing apparatus according to a fourth aspect of the present invention, the determination unit is configured to determine that the blood pressure value is suspected of being persistent hypertension when it is determined that the subject is not in the stressed state at the time of measuring the blood pressure value.

According to the fourth aspect, when the measured blood pressure value is classified as hypertension, it can be determined that the type of hypertension is suspected of being persistent hypertension defined in the guidelines for the management of hypertension in Japan.

An information processing apparatus according to a fifth aspect of the present invention further includes a location information acquisition unit configured to acquire location information indicating a location at which the blood pressure value is measured. The determination unit is configured to determine whether the type of stress-induced hypertension is a white coat hypertension, a workplace hypertension, or hypertension associated with any other location, based on the location information when it is determined that the blood pressure value is suspected of being stress-induced hypertension.

According to the fifth aspect, on the basis of the information indicating the location where the blood pressure value is measured, it is determined whether the type of the stress hypertension is white coat hypertension, workplace hypertension, or hypertension associated with other places. Therefore, the type of stress-induced hypertension can be determined more specifically.

In an information processing apparatus according to a sixth aspect of the present invention, the determination unit is configured to output information indicating a determination result.

According to the sixth aspect, the determination result of the type of blood pressure by the determination unit is output. Therefore, the subject can identify, for example, whether his/her blood pressure value corresponds to hypertension, and in the case of hypertension, whether it is stress-induced or persistent, and in the case of stress-induced hypertension, whether the hypertension is white coat hypertension, workplace hypertension, or hypertension associated with other locations.

In an information processing apparatus according to a seventh aspect of the present invention, the determination unit is configured to output information recommending measurement of a blood pressure at a normal time.

According to the seventh aspect, the information recommending that a subject measure a blood pressure at a normal time is output. If there is a suspicion of stress-induced hypertension (stress-induced hypertension) or persistent hypertension, blood pressure measurements at a normal time are recommended. Therefore, if the subject receives this message and measures the blood pressure at a normal time, the doctor can confirm the diagnosis of stress-induced hypertension or persistent hypertension from the measured value.

According to the present invention, it is possible to provide a technique capable of determining not only the blood pressure value but also the type of hypertension.

### BRIEF DESCRIPTION OF THE DRAWINGS

FIG. 1 is a block diagram schematically illustrating an example of an information processing system including an information processing apparatus according to the first embodiment.

FIG. 2 is a block diagram showing the overall configuration of the information processing system including the information processing apparatus according to the first embodiment.

FIG. 3 is a block diagram showing a configuration example of a blood pressure monitor.

FIG. 4 is a block diagram showing a configuration example of a mobile information terminal.

FIG. 5 is a block diagram showing a configuration example of a doctor terminal.

FIG. 6 is a block diagram showing a configuration example of a server.

FIG. 7 is a block diagram schematically illustrating an example of a functional configuration of the server.

FIG. 8 is a diagram showing an example of the structure of a table.

FIG. 9 is a flowchart illustrating an example of a processing procedure of the information processing system.

FIG. 10 is a flowchart illustrating an example of a processing procedure of the information processing system.

FIG. 11 is a diagram showing a relationship between a blood pressure value and a stress level related to persistent hypertension.

FIG. 12 is a diagram showing the relationship between a blood pressure value and a stress level related to stress hypertension.

FIG. 13 is a block diagram showing a configuration example of a mobile information terminal.

FIG. 14 is a block diagram schematically illustrating an example of a functional configuration of the server according to the present embodiment.

FIG. 15 is a diagram showing an example of the structure of a table.

FIG. 16 is a flowchart illustrating an example of a processing procedure of the information processing system.

FIG. 17 is a flowchart illustrating an example of a processing procedure of the information processing system.

FIG. 18 is a block diagram schematically illustrating an example of a functional configuration of the server.

FIG. 19 is a diagram showing an example of the structure of a table.

FIG. 20 is a flowchart illustrating an example of a processing procedure of the information processing system.

FIG. 21 is a flowchart illustrating an example of a processing procedure of the information processing system.

FIG. 22 is a block diagram schematically illustrating an example of a functional configuration of the server.

FIG. 23 is a diagram showing an example of the structure of a table.

FIG. 24 is a flowchart illustrating an example of the processing procedure of the information processing system.

FIG. 25 is a flowchart illustrating an example of a processing procedure of the information processing system.

FIG. 26 is a block diagram showing a configuration example of a mobile information terminal IT.

### DETAILED DESCRIPTION

Hereinafter, an embodiment according to an aspect of the present invention (hereinafter, also referred to as “present embodiment”) will be described with reference to the drawings. The embodiment described below is merely an example of the present invention in all respects. It goes without saying that various improvements and modifications can be made without departing from the scope of the present invention. That is, when carrying out the present invention, a specific configuration according to an embodiment may be adopted as appropriate. Although data appearing in the present embodiment is described in a natural language. The data is more specifically designated by a pseudo language, a command, a parameter, a machine language, or the like recognizable by a computer.

According to one embodiment of the present invention, there is provided a technique capable of determining not only a blood pressure value but also a type of hypertension.

[Application Example]

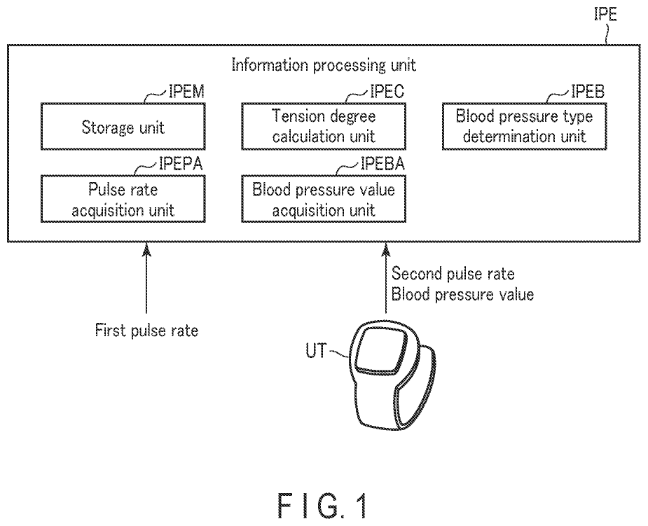

First, an example of a scene to which the present invention is applied will be described with reference to FIG. 1. FIG. 1 schematically illustrates an example of an information processing system including an information processing apparatus according to an application example.

According to the guidelines for the management of hypertension in Japan, a blood pressure value at a normal time such as home blood pressure is indispensable for determining the type of hypertension. However, there are persons whose blood pressure values at normal times cannot be acquired, such as a person who does not have a habit of measuring blood pressure at home or the like, or a patient who neglects blood pressure measurements. Therefore, an information processing system capable of determining the type of hypertension for such a person will be described.

[Configuration of Application Example]

Before describing the configuration of the information processing system, an overview of the information processing system will be described. The information processing system determines the type of blood pressure at a predetermined timing by determining the blood pressure value and the pulse rate at the predetermined timing based on a pulse rate at a normal time.

As shown in FIG. 1, the information processing system includes a user terminal UT and information processing equipment IPE.

The user terminal UT measures a blood pressure value and a pulse rate of the user (subject) and supplies the blood pressure value and the pulse rate to the information processing equipment IPE. The user terminal UT is, for example, a wristwatch-type wearable terminal. However, the user terminal UT is not limited to a wristwatch-type wearable terminal and may be appropriately selected according to the embodiment.

The information processing equipment IPE includes a pulse-rate acquisition unit IPEPA, a blood-pressure acquisition unit IPEBA, a tension degree calculation unit IPEC, a storage unit IPEM, and a blood pressure type determination unit IPEB.

The pulse rate acquisition unit IPEPA receives a user's pulse rate from the user terminal UT, another terminal, or the like.

The blood pressure value acquisition unit IPEBA receives the blood pressure value of the user from the user terminal UT, another terminal, or the like.

The tension degree calculation unit IPEC calculates a tension degree of the autonomic nerve (stress level) of the user based on the received pulse rate. The tension degree calculation unit IPEC calculates the stress level using, for example, symmetrized dot patterns (SDP) method. The stress level is a numerical value of stress undergone by the user (subject) due to mental or physical load. When the user undergoes stress, the autonomic nervous system and the endocrine system such as adrenocortical hormone may be modulated. Therefore, when the endocrine system of a user is modulated, components such as hormones contained in blood, saliva, and urine are changed. In addition, when the autonomic nervous system of the user is modulated, various physiological responses such as brain waves, facial temperature, skin surface temperature, surface potential, and eye movement change in addition to vital signs such as pulse, heartbeat, respiration, and pulse wave. The stress of the user can be calculated from the pulse rate of the user, for example. Based on such a viewpoint, the tension degree calculation unit IPEC is used to calculate the stress level of the user based on the pulse rate.

The storage unit IPEM stores the received blood pressure value and tension degree (or pulse rate) for each user.

The blood pressure type determination unit IPEB determines the type of blood pressure based on the data stored in the storage unit IPEM.

The pulse-rate acquisition unit IPEPA is an example of the “pulse-rate acquisition unit” of the present invention. The blood pressure value acquisition unit IPEBA is an example of the “blood pressure value acquisition unit” of the present invention. The tension degree calculation unit IPEC is an example of a “calculation unit” of the present invention. The blood pressure type determination unit IPEB is an example of a “determination unit” of the present invention.

[Operation of Application Example]

Next, an example of an operation in which the information processing system determines the type of blood pressure will be described.

Here, as an example, a case will be described in which the stress of the user at the time of blood pressure measurement is determined based on the pulse rate at a normal time (first pulse rate).

The user transmits the pulse rate at the normal time to the information processing equipment IPE via a discretional terminal.

The user transmits the pulse rate (second pulse rate) and the blood pressure value via the user terminal UT, for example. A second pulse rate is a pulse rate in a time period in which the blood pressure value is measured.

The tension degree calculation unit IPEC calculates a first stress level based on the first pulse rate and calculates a second stress level based on the second pulse rate.

When determining the type of the measured blood pressure, the blood pressure type determination unit IPEB determines whether or not the blood pressure value is classified as hypertension. Specifically, for example, the blood pressure type determination unit IPEB determines whether or not the blood pressure value has exceeded a threshold. In this manner, the blood pressure type determination unit IPEB determines whether or not the blood pressure value is classified as hypertension.

When the blood pressure type determination unit IPEB determines that the blood pressure value is classified as hypertension, the blood pressure type determination unit IPEB determines the magnitude of the stress at the time of measuring the blood pressure value based on the first stress level and the second stress level. Specifically, the blood pressure type determination unit IPEB determines the magnitude of the stress at the time of measuring the blood pressure value by comparing the first stress level with the second stress level.

When the blood pressure type determination unit IPEB determines that the stress at the time of blood pressure value measurement is “large”, the blood pressure type determination unit IPEB determines that the type of blood pressure is “stress-induced hypertension”. When the blood pressure type determination unit IPEB determines that the stress at the time of blood pressure value measurement is “small”, the blood pressure type determination unit IPEB determines that the type of blood pressure is “persistent hypertension”.

[Advantageous Effects of Application Example]

As described above, according to the information processing system of the application example, the stress level of the user can be determined by considering the pulse rate at a normal time and the pulse rate at the time of measuring the blood pressure value. When the stress level of the user is known, the information processing system can determine whether or not the blood pressure of the blood pressure value of the determination target is suspected of being “stress-induced hypertension”. This allows doctors to confirm the diagnosis of stress-induced hypertension or persistent hypertension.

### <1> First Embodiment

Hereinafter, a first embodiment according to the application example will be described.

<1-1> Configuration

<1-1-1> Information Processing System

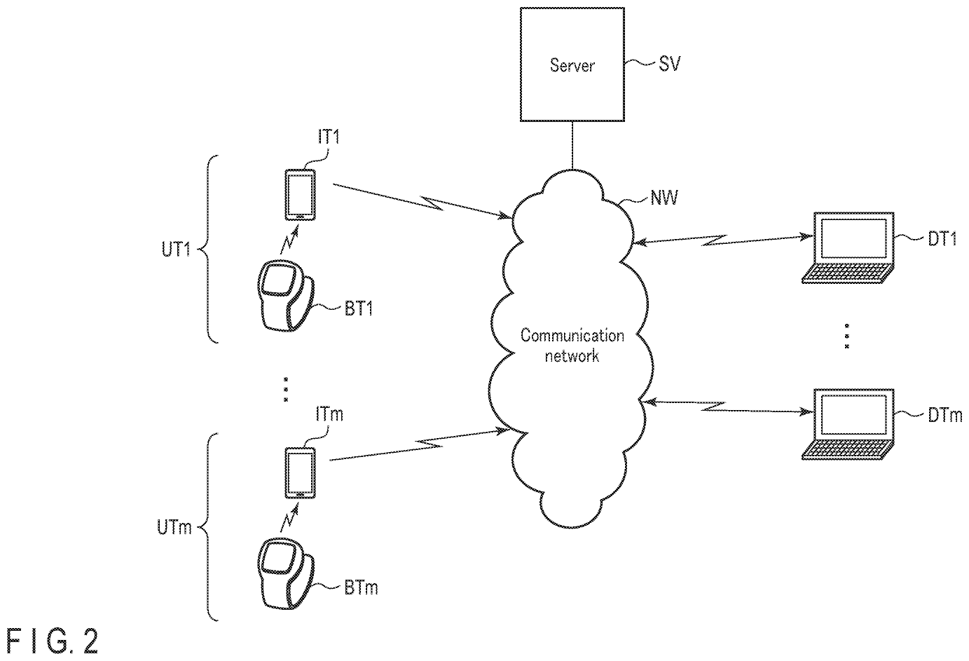

FIG. 2 is a block diagram illustrating an overall configuration of an information processing system including an information processing apparatus according to the first embodiment. As illustrated in FIG. 2, the information processing system includes, for example, a plurality of user terminals UT (UT1 to UTn in FIG. 2, where n is an arbitrary integer), a communication network NW, a server SV, and a plurality of doctor terminals DT (DT1 to DTm in FIG. 2, where m is an arbitrary integer). The user terminals UT1 to UTn, the server SV, and the doctor terminals DT1 to DTm can communicate with each other via the communication network NW. When the user terminals UT1 to UTn are not distinguished from each other, they are simply referred to as user terminals UT. Similarly, when the doctor terminals DT1 to DTm are not distinguished from each other, they are simply referred to as doctor terminals DT. The user terminal UT is an example of the “user terminal UT” of the application example. The server SV is an example of the “information processing equipment IPE” of the application example.

<1-1-1-1> User Terminal

As shown in FIG. 2, the user terminals UT1 to UTn include blood pressure monitors BT1 to BTn and mobile information terminals IT1 to ITn, respectively. When the blood pressure monitors BT1 to BTn are not distinguished from each other, they are simply referred to as the blood pressure monitor BT. Similarly, when the mobile information terminals IT1 to ITn are not distinguished from each other, they are simply referred to as the mobile information terminal IT.

<1-1-1-1-1> Blood Pressure Monitor

Before describing a specific configuration of the blood pressure monitor BT, an outline of the blood pressure monitor BT will be described. The blood pressure monitor BT is, for example, a wristwatch-type wearable terminal. The blood pressure monitor BT is worn on the wrist of a user (subject) and measures a blood pressure value and a pulse rate at a timing of the user's operation or a timing or a time interval preliminarily set. Then, the blood pressure monitor BT transmits measurement data, in which, for example, a blood pressure value of the user, a pulse rate of the user, and user information (e.g. a user ID) are associated with each other, to the mobile information terminal IT via, for example, a wireless interface. The user ID is an identifier assigned to each user. The blood pressure monitor BT may measure only a blood pressure value of the user or only a pulse rate of the user. When the blood pressure monitor BT measures only a blood pressure value of a user, for example, the measurement data includes the blood pressure value of the user and a user ID. When the blood pressure monitor BT measures only a pulse rate of the user, the measurement data includes the pulse rate of the user and the user ID, for example. Furthermore, the blood pressure monitor BT is not limited to the type of blood pressure monitor worn on the wrist and may be a type in which a cuff is wrapped around the upper arm or the like, or an installation type. The blood pressure monitors BT1 to BTn may be blood pressure monitors of different models.

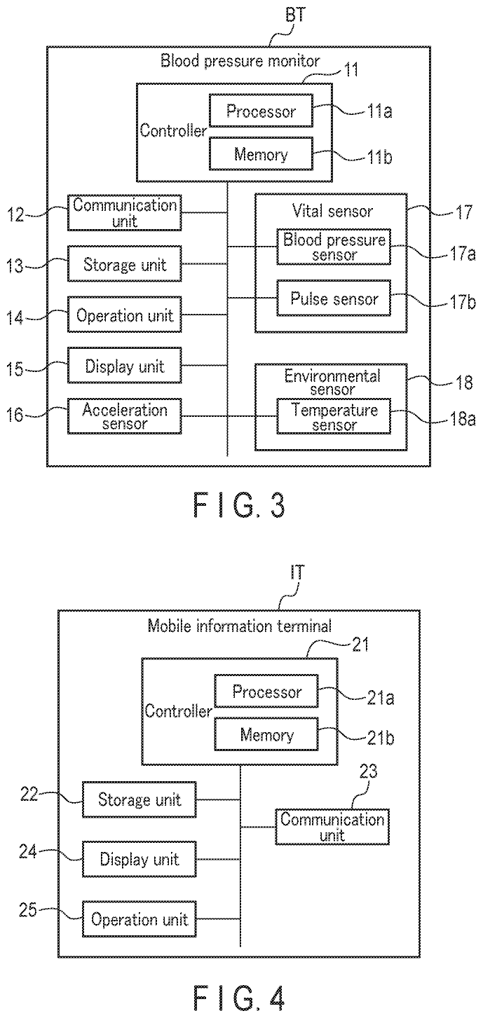

An example of a specific configuration of the blood pressure monitor BT will be described with reference to FIG. 3. FIG. 3 is a block diagram illustrating a configuration example of the blood pressure monitor BT.

As shown in FIG. 3, the blood pressure monitor BT includes a controller 11, a communication unit 12, a storage unit 13, an operation unit 14, a display unit 15, an acceleration sensor 16, a vital sensor 17, and an environmental sensor 18.

The controller 11 includes, for example, a processor 11a and a memory 11b. In the controller 11, the processor 11a executes a program using a memory 11b, thereby the controller 11 realizes various kinds of operation control, data processing, and the like. The processor 11a is, for example, a central processing unit (CPU) or micro processing unit (MPU) including an arithmetic circuit. The memory 11b includes, for example, a nonvolatile memory that stores a program executed by the processor 11a, and a volatile memory such as a random access memory (RAM) used as a working memory. The controller 11 has a clock (not shown) and can count the current date and time. The processor 11a can perform control of each unit and data processing by executing a program stored in the memory 11b or the storage unit 13. That is, the processor 11a performs operation control of each unit in accordance with an operation signal from the operation unit 14 and performs data processing for measurement data measured by the vital sensor 17 and the environmental sensor 18.

The communication unit 12 is a communication interface for communicating with the mobile information terminal IT. As the communication interface, for example, an interface adopting a short-range wireless data communication standard such as Bluetooth (Registered trademark) is used. The communication unit 12 transmits data to the mobile information terminal IT and receives data from the mobile information terminal IT. The communication by the communication unit 12 may be a wireless communication or wired communication.

The storage unit 13 stores data of a program for controlling the blood pressure monitor BT, setting data for setting various functions of the blood pressure monitor BT, measurement data measured by the acceleration sensor 16, the vital sensor 17, and the environmental sensor 18, and the like. The storage unit 13 may be used as a working memory when the program is executed.

The operation unit 14 includes, for example, an operation device such as a touch panel and operation buttons (operation keys) which are not shown. The operation unit 14 detects an operation by the user and outputs an operation signal indicating the operation contents to the controller 11. The operation unit 14 is not limited to a touch panel or operation buttons. The operation unit 14 may include, for example, a speech recognition unit that recognizes operation instructions by a speech of the user, a biometric authentication unit that authenticates a part of the living body of the user, and an image recognition unit that recognizes a facial expression or a gesture of the user by means of an image obtained by photographing the face or body of the user.

The display unit 15 includes, for example, a display screen (e.g. a liquid crystal display (LCD), or an electroluminescence (EL) display, or the like), an indicator, or the like, and displays information in accordance with a control signal from the controller 11.

The acceleration sensor 16 detects an acceleration received by the main body of the blood pressure monitor BT. For example, the acceleration sensor obtains acceleration data of three axes or six axes. The acceleration data can be used to estimate the activity amount (posture and/or motion) of a user wearing the blood pressure monitor BT. The controller 11 can associate the measurement date and time, which is based on the date and time information, with the acceleration data measured by the acceleration rate sensor 16 and output the data as measurement data.

The vital sensor 17 measures vital information of the user. The vital sensor 17 includes, for example, a blood pressure sensor 17a and a pulse sensor 17b. The blood pressure sensor 17a measures a blood pressure value of the user. The pulse sensor 17b measures the pulse rate of the user.

As the measurement data acquired by the vital sensor 17, pulse wave data, electrocardiogram data, heart rate data, body temperature data, and the like are assumed in addition to the blood pressure value and the pulse rate, and a sensor for measuring these pieces of measurement data may be provided as the vital sensor 17.

The blood pressure sensor 17a is a continuous measurement type or a non-continuous measurement type blood pressure sensor. The blood pressure sensor 17a is a blood pressure sensor capable of measuring values of blood pressure (e.g. systolic blood pressure and diastolic blood pressure). The blood pressure sensor 17a may include, but is not limited to, a beat by beat (BbB) blood pressure sensor that measures a blood pressure value for each heartbeat.

For example, as the blood pressure sensor 17a, a blood pressure sensor using an oscillometric method, a pulse transit time (PTT) method, a tonometry method, an optical method, a radio wave method, an ultrasonic method, or the like can be applied. The oscillometric method is a method in which an upper arm is pressed by a cuff and a blood pressure value is measured by an oscillation waveform in the cuff. The PTT method is a method of measuring a pulse transit time and estimating a blood pressure value from the measured pulse transit time. The tonometry method is a method in which a pressure sensor is brought into direct contact with a living body part through which an artery passes, such as a radial artery of the wrist, and a blood pressure value is measured using information detected by the pressure sensor. The optical method, the radio wave method, or the ultrasonic method is a method in which light, radio wave, or an ultrasonic wave is applied to a blood vessel and a blood pressure value is measured from a reflected wave thereof.

The environmental sensor 18 includes a sensor that measures environmental information around the user and acquires measured environmental data. In the configuration example shown in FIG. 3, the environmental sensor 18 includes, for example, a temperature sensor 18a. The environmental sensor 18 may include a sensor that measures temperature, humidity, sound, light, or the like in addition to temperature. The environmental sensor 18 may include a sensor that measures information in an environment (environment data) that is assumed to be directly or indirectly associated with a change in blood pressure value. The controller 11 can associate the measurement date and time, which is set based on the date and time information, with the measurement data measured by the environmental sensor 18 and output the data as measurement data (environment data).

<1-1-1-1-2> Mobile Information Terminal

Before describing a specific configuration of the mobile information terminal IT, an outline of the mobile information terminal IT will be described. The mobile information terminal IT is, for example, a smart device (typically, a smartphone or a tablet terminal). The mobile information terminal IT receives measurement data transmitted from the blood pressure monitor BT and transfers the measurement data to the server SV via a communication network NW. In the mobile information terminal IT, for example, application software (a program) for managing measurement data may be installed. The mobile information terminals IT1 to ITn may be terminals of different models. When a user ID is not associated with measurement data received from the blood pressure monitor BT, the mobile information terminal IT may associate the user ID with the measurement data received from the blood pressure monitor BT. The user ID may be stored in a storage unit 22 or a memory 21b.

An example of a specific structure of the mobile information terminal IT will be described with reference to FIG. 4. FIG. 4 is a block diagram illustrating a configuration example of the mobile information terminal IT.

As shown in FIG. 4, the mobile information terminal IT includes a controller 21, a storage unit 22, a communication unit 23, a display unit 24, an operation unit 25, and the like.

The controller 21 includes, for example, the processor 21a and the memory 21b. Since the basic configuration of the controller 21 is the same as that of the controller 11, a detailed description thereof will be omitted.

The storage unit 22 includes, for example, a semiconductor memory or a magnetic disk. The storage unit 22 may store a program executed by the processor 21a of the controller 21. The storage unit 22 may store measurement data supplied from the blood pressure monitor BT. The storage unit 22 may also store display data to be displayed on the display unit 24.

The communication unit 23 is a communication interface for communicating with the blood pressure monitor BT and the server SV. The communication unit 23 receives data from the blood pressure monitor BT or transmits operation instructions to the blood pressure monitor BT. The communication by the communication unit 23 may be a wireless communication or wired communication. Furthermore, the communication unit 23 transmits data to the server SV or receives data from the server SV via the network NW. The communication by the communication unit 23 may be a wireless communication or wired communication. In the present embodiment, the network NW is described assuming, for example, that it is the Internet or the like, but the network NW is not limited thereto, and may be another type of network such as a LAN or may be one-to-one communication using a communication cable such as a USB cable.

The display unit 24 includes a display screen (e.g. an LCD or an EL display). The display unit 24 is controlled by the controller 21 to display contents on the display screen.

The operation unit 25 transmits an operation signal corresponding to an operation by the user to the controller 21. The operation unit 25 is, for example, a touch panel provided on the display screen of the display unit 24. The operation unit 25 is not limited to a touch panel, and may be an operation button, a keyboard, a mouse, or the like. The operation unit 25 may include a speech recognition unit that recognizes operation instructions by a speech of the user, a biometric authentication unit that authenticates a part of the living body of the user, an image recognition unit that recognizes a facial expression or a gesture of the user, or the like.

When the blood pressure monitor BT cannot transmit measurement data to the mobile information terminal IT, the mobile information terminal IT may transmit the blood pressure value and the pulse rate manually entered by the user to the server SV.

<1-1-1-2> Doctor Terminal

Before describing a specific configuration of a doctor terminal DT, an outline of the doctor terminal DT will be described. The doctor terminal DT is, for example, a fixed personal computer, a portable notebook personal computer, or a tablet terminal. The doctor terminal DT can transmit and receive data to and from the server SV by using, for example, a browser. Specifically, the doctor terminal DT can use a browser to transmit information on the user to the server SV and to display the information transmitted from the server SV. The doctor terminals DT1 to DTm may be terminals of different models. The doctor terminal DT may receive measurement data from the blood pressure monitor BT and perform various processes.

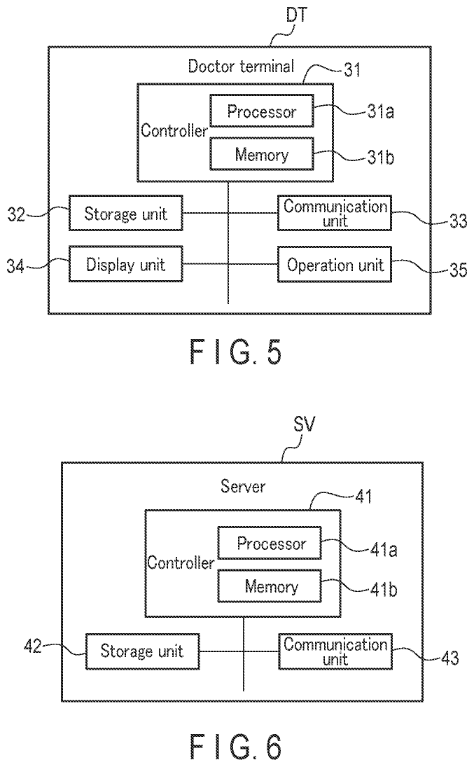

An example of a specific configuration of the doctor terminal DT will be described with reference to FIG. 5. FIG. 5 is a block diagram illustrating a configuration example of the doctor terminal DT.

As shown in FIG. 5, the doctor terminal DT includes a controller 31, a storage unit 32, a communication unit 33, a display unit 34, an operation unit 35, and the like.

The controller 31 includes, for example, a processor 31a and a memory 31b. Since the basic configuration of the controller 31 is the same as that of the controller 11, a detailed description thereof will be omitted.

The storage unit 32 includes, for example, a magnetic disk, a semiconductor memory, an optical disk, a magneto-optical disk, or the like. The storage unit 32 may store a program executed by the processor 31a of the controller 31.

The communication unit 33 is a communication interface for communicating with the server SV. The communication unit 33 transmits data to the server SV or receives data from the server SV via a network NW. The communication by the communication unit 33 may be a wireless communication or wired communication. In the present embodiment, the communication unit 33 is described assuming that it communicates with the server SV via another type of network such as a LAN. However, the present invention is not limited thereto, and may include a communication unit that performs communication serially using a communication cable.

The display unit 34 includes a display screen (e.g. an LCD or an EL display). The display unit 34 is controlled by the controller 31 to display contents on the display screen.

The operation unit 35 transmits an operation signal corresponding to an operation by the user to the controller 31. The operation unit 35 is, for example, a touch panel provided on the display screen of the display unit 34. The operation unit 35 is not limited to a touch panel, and may be an operation button, a keyboard, a mouse, or the like. The operation unit 35 may include a speech recognition unit that recognizes operation instructions from a speech of the user, a biometric authentication unit that authenticates a part of the living body of the user, an image recognition unit that recognizes a facial expression or a gesture of the user, or the like.

<1-1-1-3> Server

Before describing a specific configuration of the server SV, an outline of the server SV will be described. The server SV is a server computer. In the present embodiment, it is assumed that the server SV is a general-purpose computer device in which a program (software) is installed so as to perform processing described below. The server SV stores the measurement data transmitted from the user terminal UT. The server SV may transmit measurement data of the user in response to access from a doctor terminal DT installed in a medical institution, for example, in order to provide health guidance or diagnosis of the user. Examples of functions realized by the server SV will be described later.

An example of a specific configuration of the server SV will be described with reference to FIG. 6. FIG. 6 is a block diagram illustrating a configuration example of the server SV.

As shown in FIG. 6, the server SV includes a controller 41, a storage unit 42, and a communication unit 43.

The controller 41 includes, for example, a processor 41a and a memory 41b. Since the basic configuration of the controller 41 is the same as that of the controller 11, a detailed description thereof will be omitted.

The storage unit 42 includes, for example, a magnetic disk, a semiconductor memory, an optical disk, a magneto-optical disk, or the like. The storage unit 42 stores various pieces of measurement data acquired from the user terminal UT. The storage unit 42 may store a program executed by the processor 41a of the controller 41.

The communication unit 43 is a communication interface for communicating with the user terminal UT or the doctor terminal DT. The communication unit 43 transmits data to the user terminal UT or the doctor terminal DT via the network NW or receives data from the user terminal UT or the doctor terminal DT via the network NW. The communication by the communication unit 43 may be a wireless communication or wired communication.

<1-1-2> Functional Configuration of Server

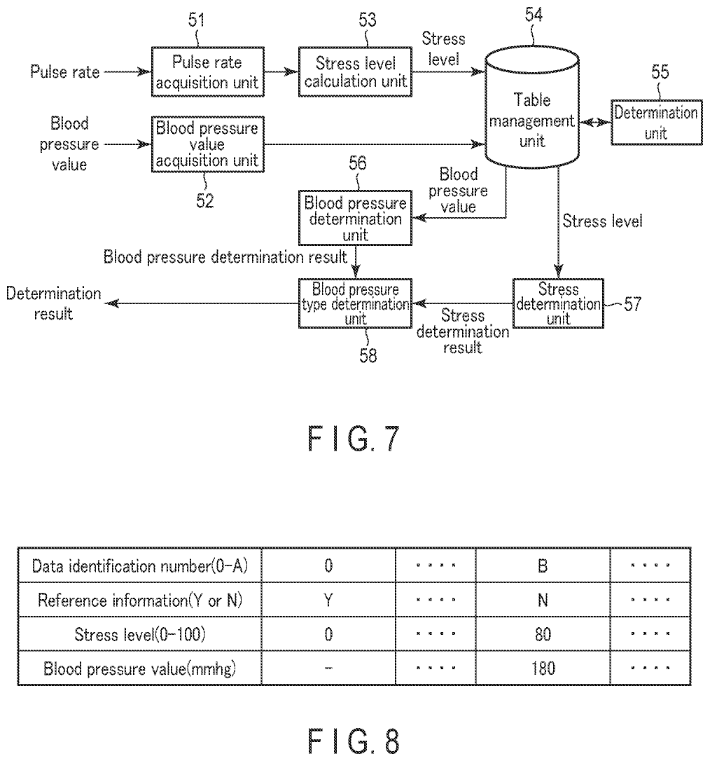

Next, an example of a functional configuration of the server SV according to the present embodiment will be described with reference to FIG. 7. FIG. 7 is a block diagram schematically illustrating an example of a functional configuration of the server SV according to the present embodiment.

The controller 41 of the server SV loads the program stored in the storage unit 42 into the memory 41b. Then, the controller 41 causes the processor 41a to interpret and execute the program loaded in the memory 41b and controls each component. Thereby, as shown in FIG. 7, the server SV according to the present embodiment functions as a computer including a pulse rate acquisition unit 51, a blood pressure value acquisition unit 52, a stress level calculation unit 53, a table management unit 54, a determination unit 55, a blood pressure determination unit 56, a stress determination unit 57, and a blood pressure type determination unit 58. The pulse rate acquisition unit 51 is an example of the “pulse rate acquisition unit IPEPA” of the application example. The blood pressure value acquisition unit 52 is an example of the “blood pressure value acquisition unit IPEBA” of the application example. The stress level calculation unit 53 is an example of the “tension degree calculation unit IPEC” of the application example. The table management unit 54 is an example of the “storage unit IPEM” of the application example. The determination unit 55, the blood pressure determination unit 56, the stress determination unit 57, and the blood pressure type determination unit 58 are examples of the “blood pressure type determination unit IPEB” of the application example.

The pulse rate acquisition unit 51 receives a pulse rate via the network NW and supplies the pulse rate to the stress level calculation unit 53.

The stress level calculation unit 53 calculates a stress level (tension degree) based on the pulse rate. Specifically, the stress level calculation unit 53 calculates a stress level associated with a user ID based on the pulse rate associated with the user ID. After calculating the stress level from the pulse rate of the user, the stress level calculation unit 53 supplies the stress level to the table management unit 54.

The blood pressure value acquisition unit 52 receives' a blood pressure value via the network NW and supplies the blood pressure value to the table management unit 54.

The table management unit 54 includes a table for each user. By managing the table for each user, it is possible to appropriately manage information of a plurality of subjects. The table is loaded in, for example, the memory 41b or the storage unit 42 of the server SV. The table stores, for example, blood pressure values received via the network NW and stress levels received from the stress level calculation unit 53. A specific structure example of the table will be described later. The table management unit 54 can display the information on the mobile information terminal IT or the doctor terminal DT in response to instructions from the user via the mobile information terminal IT or the doctor terminal DT.

The determination unit 55 determines the content of the data stored in the table management unit 54 based on a command from the user and controls the operation of the table management unit 54.

The blood pressure determination unit 56 determines whether or not the blood pressure value supplied from the table management unit 54 has exceeded a threshold. Then, the blood pressure determination unit 56 supplies the determination result (blood pressure determination result) to the blood pressure type determination unit 58.

The stress determination unit 57 determines the stress of the user to be determined based on the stress level supplied from the table management unit 54. Specifically, the stress determination unit 57 compares the stress level of the user at a normal time with the stress level at the time of blood pressure measurement. Then, the stress determination unit 57 determines whether or not the stress level at the time of blood pressure measurement exceeds a threshold with respect to the stress level at a normal time. The stress determination unit 57 supplies the determination result (stress determination result) to the blood pressure type determination unit 58.

The blood pressure type determination unit 58 determines the type of blood pressure based on the stress determination result supplied from the stress determination unit 57 and the blood pressure determination result supplied from the blood pressure determination unit 56. Then, the blood pressure type determination unit 58 outputs the determination result.

<1-1-3> Table Structure Example

Next, an example of the structure of the table will be described with reference to FIG. 8. FIG. 8 is a diagram illustrating an example of a structure of a table. For simplicity, the structure of the table will be described focusing on one user.

As illustrated in FIG. 8, the table stores, for example, a data identification number, reference information, a stress level, and a blood pressure value for each piece of user information (e.g. a user ID) included in measurement data.

Here, the reference information will be described. The reference information is information indicating whether or not the stress level becomes a reference value in the blood pressure type determination operation described later. In the blood pressure type determination operation, the information processing system determines a pulse rate (stress level) at the time of blood pressure measurement based on a pulse rate (stress level) at a normal time, and determines the stress state of the user (subject). Therefore, it is necessary to determine which pulse rate is the pulse rate at the normal time. Then, the user needs to set the reference information of the pulse rate at the normal time to “Y” and set the reference information of the pulse rate at times other than the normal time to “N”. That is, the reference information of the stress level that is a reference value is set to “Y”, and the reference information of the stress level that is not the reference is set to “N”.

Here, a method of associating the reference information with the measurement data (pulse rate) will be described.

(Method 1)

For example, when a doctor manually enters the pulse rate at the normal time of the subject via the doctor terminal DT, the doctor enters the pulse rate at the normal time of the subject via the operation unit 35 and sets the reference information to “Y”. Accordingly, the pulse rate and the reference information are associated with each other.

(Method 2)

When the pulse rate is measured by the blood pressure monitor BT, the user enters reference information (at a normal time or not) via the operation unit 14. The controller 11 further associates the reference information with the measurement data (pulse rate) based on the input from the operation unit 14.

(Method 3)

The user enters information on the reference information (at a normal time or not) via the operation unit 25 for the measurement data (pulse rate) transferred from the blood pressure monitor BT to the mobile information terminal IT. The controller 21 further associates the reference information with the measurement data (pulse rate) based on the input from the operation unit 25.

Methods 1 to 3 described above are examples, and the method of associating the reference information with the measurement data can be suitably applied.

As described above, the stress level is associated with a user ID. The blood pressure value is associated with the user ID. Therefore, upon receiving various types of information, the table management unit 54 stores data in the table associated with the user ID.

In addition, the blood pressure information may not be stored in a column (the vertical axis direction in FIG. 8) related to the stress level at a normal time (the stress level when the reference information is Y).

For example, the table management unit 54 can output a corresponding blood pressure value and a corresponding stress level from the user ID, the data identification number, the reference information, and the like.

The user ID is constituted by a combination of discretional numbers and characters. The data identification numbers are assigned in order from 0, for example, but are not limited thereto. For example, the data identification number may be constituted by a combination of discretional numbers or characters. The stress level is expressed as being between 0 and 100, for example, but is not limited thereto. The stress level can be appropriately changed by a calculation method, or the like. The stress level according to the present embodiment is determined to be large when the stress level is, for example, 51 to 100.

<1-2> Operation

<1-2-1> Measurement Data Storage Operation

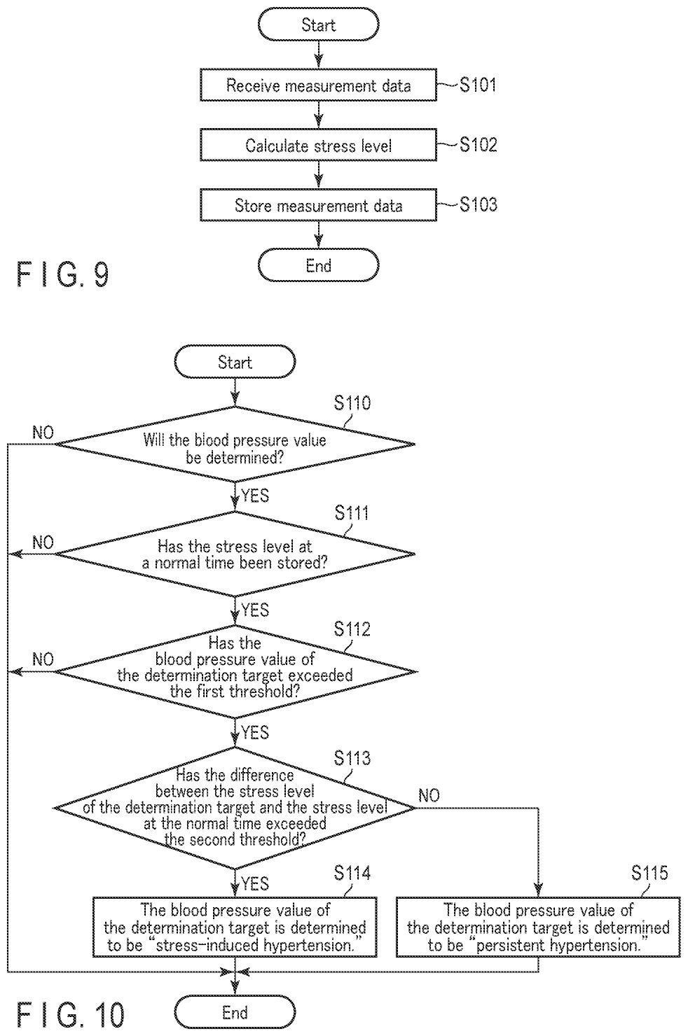

Next, an example of a measurement data storage operation of the information processing system including the information processing apparatus according to the first embodiment will be described with reference to FIG. 9. FIG. 9 is a flowchart illustrating an example of a processing procedure of the information processing system. The processing procedure described below is merely an example, and each processing may be changed as appropriate. In the processing procedure described below, steps can be omitted, replaced, and added as appropriate according to the embodiment.

[Step S101]

The server SV receives measurement data via a network NW. Here, an example of a case of receiving measurement data will be described.

(Case 1)

A case where the server SV receives only a pulse rate at a normal time from the blood pressure monitor BT will be described. For example, the user may measure only the pulse rate at a normal time by the blood pressure monitor BT. The operator of the blood pressure monitor BT supplies the pulse rate at the normal time to the server SV via the blood pressure monitor BT. In this way, the server SV (pulse rate acquisition unit 51) receives the pulse rate at the normal time. At this time, the operator of the blood pressure monitor BT sets reference information of the pulse rate at the normal time to “Y” in the blood pressure monitor BT or the mobile information terminal IT, and further associates the user ID with the pulse rate.

(Case 2)

A case where the server SV receives a pulse rate at a normal time of the user (subject) who does not have the blood pressure monitor BT will be described. The user who does not have the blood pressure monitor BT measures a pulse rate at a normal time using, for example, another terminal or by the user himself/herself. For example, the user transmits or manually enters the pulse rate at the normal time to the mobile information terminal IT or the doctor terminal DT. The user supplies a pulse rate at a normal time to the server SV via the mobile information terminal IT or the doctor terminal DT. In this way, the server SV (pulse rate acquisition unit 51) receives the pulse rate at the normal time. At this time, in the mobile information terminal IT or the doctor terminal DT, the reference information of the pulse rate at the normal time is set to “Y”, and the user ID is associated with the pulse rate.

(Case 3)

A case where the server SV receives the pulse rate and the blood pressure value for determination from the blood pressure monitor BT will be described. For example, the user may measure the pulse rate and the blood pressure value by the blood pressure monitor BT. The user supplies the pulse rate and the blood pressure value to the server SV via the blood pressure monitor BT. In this way, the server SV (pulse rate acquisition unit 51 and blood pressure value acquisition unit 52) receives the pulse rate and the blood pressure value. At this time, the user sets the reference information of the pulse rate and the blood pressure value to “N” in the blood pressure monitor BT or the mobile information terminal IT, and further associates the user ID with the pulse rate and the blood pressure value.

(Case 4)

A case where the server SV receives a pulse rate and a blood pressure value for determination of the user (subject) who does not have the blood pressure monitor BT will be described. The user who does not have the blood pressure monitor BT measures a pulse rate and a blood pressure value using, for example, another terminal provided in a medical institution or the like. For example, the user transmits or manually enters the measured pulse rate and blood pressure value to the mobile information terminal IT or the doctor terminal DT. The user supplies the pulse rate and the blood pressure value to the server SV via the mobile information terminal IT or the doctor terminal DT. In this way, the server SV (pulse rate acquisition unit 51 and blood pressure value acquisition unit 52) receives the pulse rate and the blood pressure value for determination. At this time, the user sets the reference information of the pulse rate at the normal time to “Y” in the mobile information terminal IT or the doctor terminal DT, and further associates the user ID with the pulse rate.

Although the case of receiving measurement data has been described, the present invention is not limited thereto. In Cases 1 to 4, the operator of each terminal is not limited to the user and may be, for example, a doctor.

[Step S102]

When the server SV receives the measurement data via the network NW, the stress level calculation unit 53 calculates a stress level based on the pulse rate.

[Step S103]

The table management unit 54 stores the reference information, the stress level, and the blood pressure value in the table based on the user ID.

Here, an example of a flow in which data is stored in the table will be briefly described.

When the table management unit 54 receives the blood pressure value or the stress level, the reference information, the stress level, and the blood pressure value are stored in a table corresponding to the user ID. At this time, if there is no table related to the user ID, the table management unit 54 generates a table related to the user ID. Then, the table management unit 54 stores the reference information, the stress level, and the blood pressure value in the column of the data identification number “0”. When there is a table related to the user ID, the table management unit 54 increments the latest data identification number by one, generates a new column, and stores the reference information, the stress level, and the blood pressure value.

<1-2-2> Blood Pressure Type Determination Operation

Next, an example of the blood pressure type determination operation of the information processing system including the information processing apparatus according to the first embodiment will be described with reference to FIG. 10. FIG. 10 is a flowchart illustrating an example of a processing procedure of the information processing system. The processing procedure described below is merely an example, and each processing may be changed as appropriate. In the processing procedure described below, steps can be omitted, replaced, and added as appropriate according to the embodiment.

[Step S110]

The controller 41 of the server SV determines whether to determine the type of blood pressure. The instruction as to whether to determine the type of blood pressure is received from, for example, the mobile information terminal IT or the doctor terminal DT. The diagnostician or the like of the user can select the blood pressure value of a determination target via the mobile information terminal IT or the doctor terminal DT by referring to the above-described table, for example.

[Step S111]

When the determination unit 55 judges that the type of blood pressure will be determined (Yes in step S110), the determination unit 55 determines whether or not the stress level at the normal time which will be a reference value is stored in the table. When the determination unit 55 determines that the stress level at the normal time is not stored in the table (No in step S111), the determination unit 55 stops the blood pressure type determination operation.

[Step S112]

When the determination unit 55 determines that the stress level at the normal time is stored in the table (Yes in step S111), the blood pressure determination unit 56 determines whether or not the blood pressure value of the determination target has exceeded a first threshold. Specifically, the blood pressure determination unit 56 determines whether or not the blood pressure value of the determination target has exceeded the first threshold.

The first threshold is a value for determining that the blood pressure value of the determination target is classified as hypertension. That is, when the blood pressure value of the determination target has exceeded the first threshold, it is determined that the blood pressure is classified as hypertension, and when the blood pressure value of the determination target has not exceeded the first threshold, it is determined that the blood pressure is not classified as hypertension. The first threshold is stored in, for example, the memory 41b of the server SV or the storage unit 42. For example, the doctor can arbitrarily set the first threshold via the doctor terminal DT.

When the blood pressure determination unit 56 determines that the blood pressure value of the determination target has not exceeded the first threshold (No in step S112), the blood pressure determination unit 56 determines that the blood pressure value of the determination target is not classified as hypertension and ends the blood pressure type determination operation.

[Step S113]

When it is determined by the blood pressure determination unit 56 that the blood pressure value of the determination target has exceeded the first threshold (Yes in step S112), the stress determination unit 57 determines whether or not the difference between the stress level associated with the blood pressure value of the determination target (the stress level of the determination target) and the stress level at the normal state has exceeded a second threshold.

The second threshold is a value for determining the magnitude of the stress applied to the user at the time of blood pressure value measurement. That is, if the difference between the stress level at the time of blood pressure measurement and the stress level at a normal time has exceeded the second threshold, it is determined that the user is stressed at the time of blood pressure measurement, and if the difference between the stress level at the time of blood pressure measurement and the stress level at the normal time has not exceeded the second threshold, it is determined that the user is not stressed at the time of blood pressure measurement. The second threshold is stored in, for example, the memory 41b or the storage unit 42 of the server SV. For example, the doctor can discretionally set the second threshold via the doctor terminal DT.

Only when the blood pressure value of the determination target is classified as hypertension, the process of determining the type of hypertension is performed. Therefore, when the measured blood pressure value is not classified as hypertension, the process of determining the type of hypertension is omitted, and the processing load of the server SV is reduced accordingly.

[Step S114]

When it is determined by the stress determination unit 57 that the difference between the stress level of the determination target and the stress level in the normal state has exceeded the second threshold (Yes in step S113), the blood pressure type determination unit 58 determines that the blood pressure of the determination target blood pressure value is suspected of being “stress hypertension”, and outputs the determination result. The determination result may be stored in the memory 41b or the storage unit 42 of the server SV, or may be output to the mobile information terminal IT or the doctor terminal DT.

[Step S115]

When it is determined by the stress determination unit 57 that the difference between the stress level of the determination target and the stress level in the normal state has not exceeded the second threshold (No in step S113), the blood pressure type determination unit 58 determines that the blood pressure of the determination target blood pressure value is suspected of being “persistent hypertension” and outputs the determination result. The determination result may be stored in the memory 41b or the storage unit 42 of the server SV, or may be output to the mobile information terminal IT or the doctor terminal DT.

In the blood pressure type determination operation, the blood pressure type determination unit 58 may output information recommending that the subject measure the blood pressure in a normal state.

<1-3> Advantageous Effects

According to the embodiment described above, the information processing system can determine the type of blood pressure by determining a discretional blood pressure value and stress level based on the stress level at a normal time.

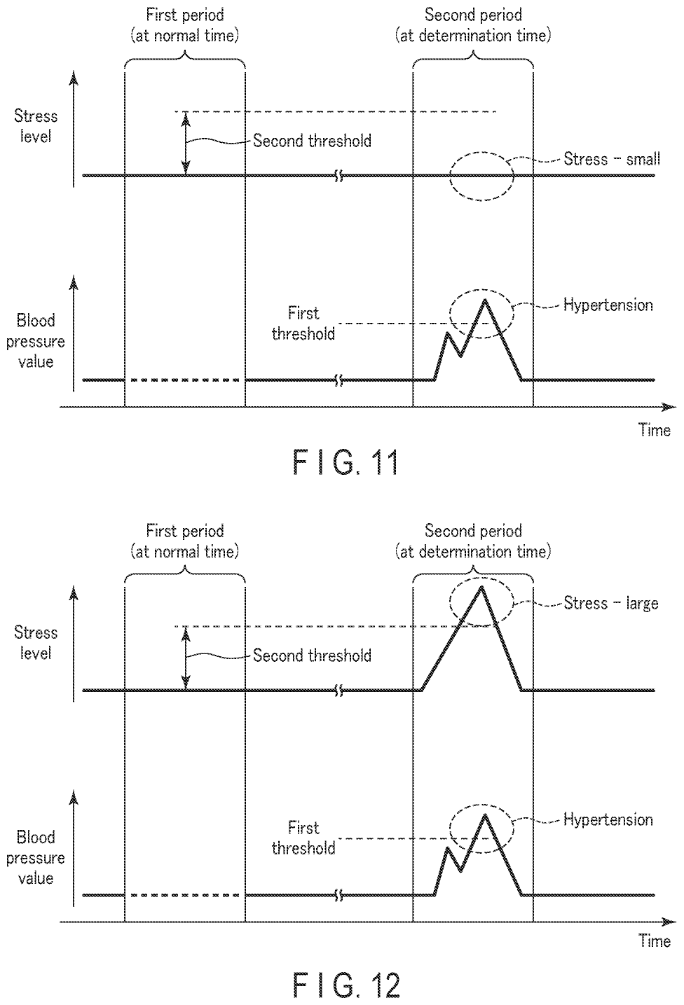

In order to facilitate understanding of the effect of the first embodiment, a specific example of the blood pressure type determination operation will be described with reference to FIGS. 11 and 12. FIG. 11 is a graph showing the relationship between the blood pressure value and the stress level associated with persistent hypertension. FIG. 12 is a graph showing the relationship between the blood pressure value and the stress level associated with stress-induced hypertension.

In the specific examples shown in FIGS. 11 and 12, in the first period (at a normal time), the user does not measure the blood pressure value but measures only the pulse rate. Then, the user measures the blood pressure value and the pulse rate in the second period. A case of determining the type of blood pressure of the blood pressure value measured in the second period under the above-described conditions will be described.

As shown in FIG. 11, in the case of persistent hypertension, the blood pressure value has exceeded the first threshold, but the difference between the stress level in the second period and the stress level in the first period has fallen below the second threshold. On the other hand, as shown in FIG. 12, in the case of stress hypertension, the blood pressure value has exceeded the first threshold, and the difference between the stress level in the second period and the stress level in the first period has exceeded the second threshold.

As shown in FIGS. 11 and 12, the blood pressure values in the second period are the same, but the stress levels are different. When the information processing system refers only to the blood pressure value in the second period without considering the stress level, the type of blood pressure cannot be determined.

However, the information processing system according to the first embodiment can determine the stress state of the user by referring to the stress level at the time of blood pressure measurement (second period). As a result, the information processing system according to the first embodiment can appropriately determine the type of blood pressure.

According to the guidelines for the management of hypertension in Japan, a blood pressure value at a normal time such as home blood pressure is indispensable for determining the type of hypertension. However, according to the first embodiment, it is possible to determine the type of hypertension even for a person whose blood pressure value cannot be acquired at a normal time, such as a person who does not have a habit of measuring blood pressure at home or the like or a patient who neglects blood pressure measurement.

In addition, when the above-mentioned stress hypertension or persistent hypertension is suspected, blood pressure measurement at a normal time is recommended. Therefore, if the subject receives this message and measures the blood pressure at a normal time, the doctor can confirm the diagnosis of stress-induced hypertension or persistent hypertension from the measured value.

### <2> Second Embodiment

A second embodiment will be described. In the second embodiment, a method of more specifically identifying the type of blood pressure by further considering location information (measurement location information) in the blood pressure type determination operation will be described. The basic configuration and basic operation of the information processing system including the information processing apparatus according to the second embodiment are the same as those of the information processing system including the information processing apparatus according to the first embodiment described above. Therefore, descriptions of the matters described in the first embodiment and matters that can be easily analogized from the first embodiment will be omitted.

[2-1] Configuration

<2-1-1> User Terminal

The user terminal UT also acquires location information when measuring the blood pressure value and the pulse rate of the user (subject). For example, a location detector of the mobile information terminal IT acquires the location information of the user. Then, the controller 21 of the mobile information terminal IT further associates the location information with the measurement data (for example, the blood pressure value, the pulse rate, and the user ID). The blood pressure monitor BT may acquire the location information. In this case, the controller 11 of the blood pressure monitor BT associates the location information with the measurement data.

Hereinafter, a case where the mobile information terminal IT includes a location detector will be described as an example.

<2-1-2> Mobile Information Terminal

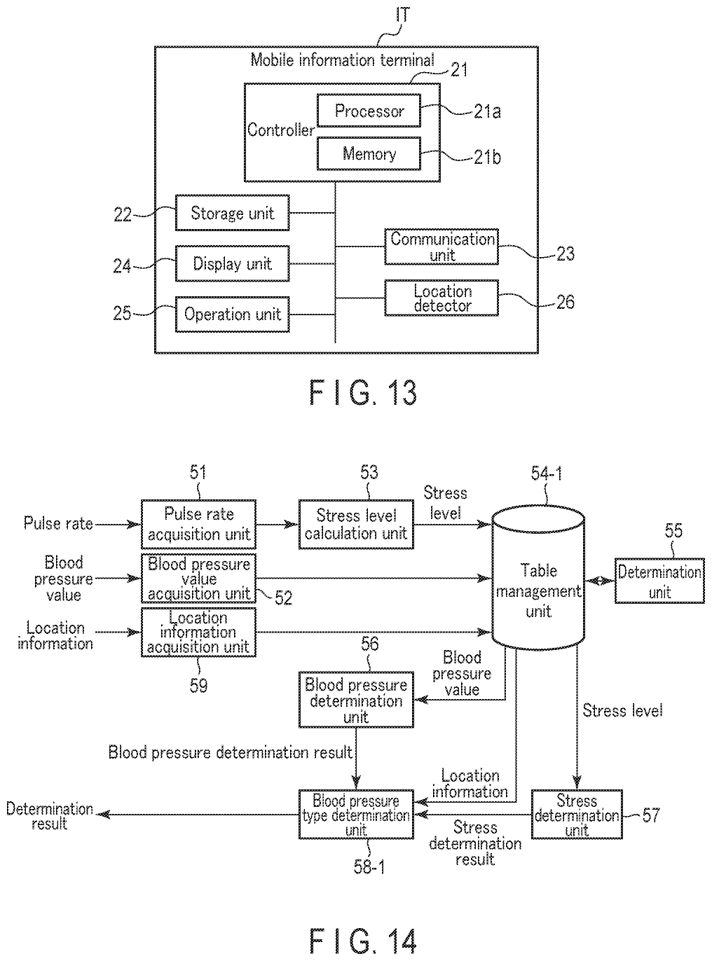

An example of a specific structure of the mobile information terminal IT will be described with reference to FIG. 13. FIG. 13 is a block diagram illustrating a configuration example of the mobile information terminal IT.

As illustrated in FIG. 13, the mobile information terminal IT includes a controller 21, a storage unit 22, a communication unit 23, a display unit 24, an operation unit 25, a location detector 26, and the like.

The location detector 26 includes, for example, a GPS (Global Positioning System), operates according to a control signal from the controller 21, and can detect the location of the mobile information terminal IT from information obtained from a GPS satellite.

<2-1-3> Functional Configuration of Server

Next, an example of a functional configuration of the server SV according to the present embodiment will be described with reference to FIG. 14. FIG. 14 is a block diagram schematically illustrating an example of a functional configuration of the server SV according to the present embodiment. This embodiment is different from the first embodiment in that the table stores location information.

The controller 41 of the server SV loads a program stored in the storage unit 42 into the memory 41b. Then, the controller 41 causes the processor 41a to interpret and execute the program loaded in the memory 41b and controls each component. Accordingly, the server SV according to the present embodiment functions as a computer including the pulse rate acquisition unit 51, the blood pressure value acquisition unit 52, the stress level calculation unit 53, a table management unit 54-1, the determination unit 55, the blood pressure determination unit 56, the stress determination unit 57, a blood pressure type determination unit 58-1, and a location information acquisition unit 59.

The location information acquisition unit 59 receives the location information via the network NW and supplies the location information to the table management unit 54-1.

The table management unit 54-1 includes a table for each user. The table is loaded in, for example, the memory 41b or the storage unit 42 of the server SV. The table stores, for example, blood pressure values, location information, and stress levels. A specific structure example of the table will be described later. The table management unit 54-1 can display the information on the mobile information terminal IT or the doctor terminal DT in response to instructions from the user via the mobile information terminal IT or the doctor terminal DT.

The blood pressure type determination unit 58-1 determines the type of blood pressure based on the location information supplied from the table management unit 54-1, the blood pressure determination result supplied from the blood pressure determination unit 56, and the stress determination result supplied from the stress determination unit 57. Then, the blood pressure type determination unit 58-1 outputs the determination result.

<2-1-4> Table Structure Example

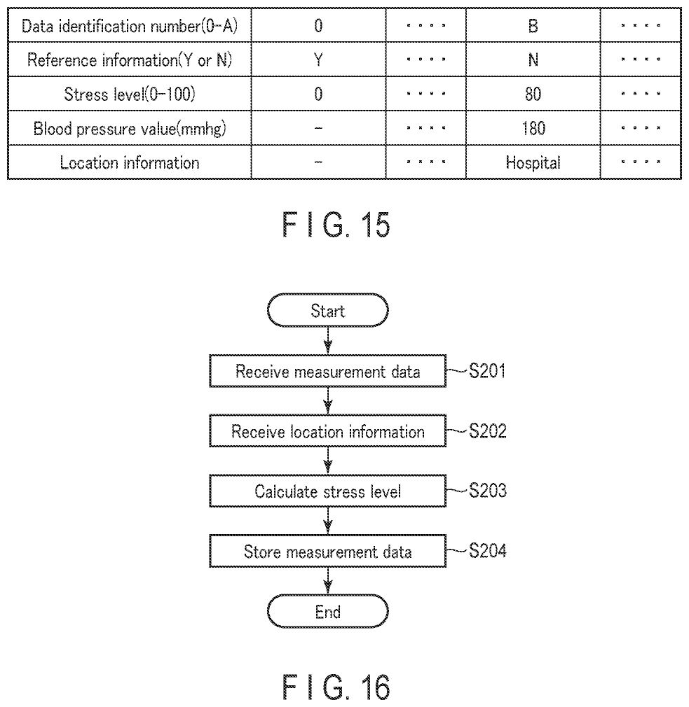

Next, an example of the structure of the table will be described with reference to FIG. 15. FIG. 15 is a diagram illustrating an example of a structure of a table. For simplicity, the structure of the table will be described focusing on one user.

As illustrated in FIG. 15, the table stores, for example, a data identification number, reference information, a stress level, a blood pressure value, and location information for each piece of user information (for example, a user ID) included in measurement data.

The location information is information for ascertaining a measurement place of the blood pressure value and the pulse rate of the user (subject). In FIG. 15, the name of a place is shown as an example, but the present invention is not limited thereto, and an address, latitude and longitude, or the like may be used.

It should be noted that the blood pressure information and the location information may not be stored in a column related to the stress level at a normal time (the stress level when the reference information is Y).

Here, a method of associating the location information with the measurement data (pulse rate and blood pressure value) will be described. Since the location information is not necessary for the pulse rate (stress level) at the normal time, it is not necessary to associate the location information with the pulse rate (stress level) at the normal time.

(Method 1)

When the mobile information terminal IT receives the measurement data (blood pressure value and pulse value) from the blood pressure monitor BT in the communication unit 23, the location detector 26 of the mobile information terminal IT acquires the location information of the user (subject). Then, the controller 21 of the mobile information terminal IT associates the location information with the measurement data.

(Method 2)

When the measurement data (blood pressure value and pulse value) is received by the communication unit 33, the doctor terminal DT inputs the location information of the user (subject) via the operation unit 35. Then, the controller 31 of the doctor terminal DT associates the location information with the measurement data.

Method 1 and Method 2 described above are examples, and the method of associating the location information with the measurement data can be appropriately applied.

<2-2> Operation

<2-2-1> Measurement Data Storage Operation

Next, an example of a measurement data storage operation of the information processing system including the information processing apparatus according to the second embodiment will be described with reference to FIG. 16. FIG. 16 is a flowchart illustrating an example of a processing procedure of the information processing system. The processing procedure described below is merely an example, and each processing may be changed as appropriate. In the processing procedure described below, steps can be omitted, replaced, and added as appropriate according to the embodiment.

The operation of storing a pulse rate at the normal time in the server SV is the same as the operation described in FIG. 9. Here, the operation of storing the pulse rate and the blood pressure value to be subjected to the blood pressure type determination operation in the server SV will be described.

[Step S201]

The operation in step S201 is the same as the operation in step S101 in FIG. 9 (in particular, cases 3 and 4).

[Step S202]

The location information acquisition unit 59 receives location information via the network NW.

[Step S203]

The operation in step S203 is the same as the operation in step S102 in FIG. 9.

[Step S204]

The table management unit 54-1 stores the reference information, the stress level, the blood pressure value, and the location information in a table based on the user ID.

<2-2-2> Blood Pressure Type Determination Operation

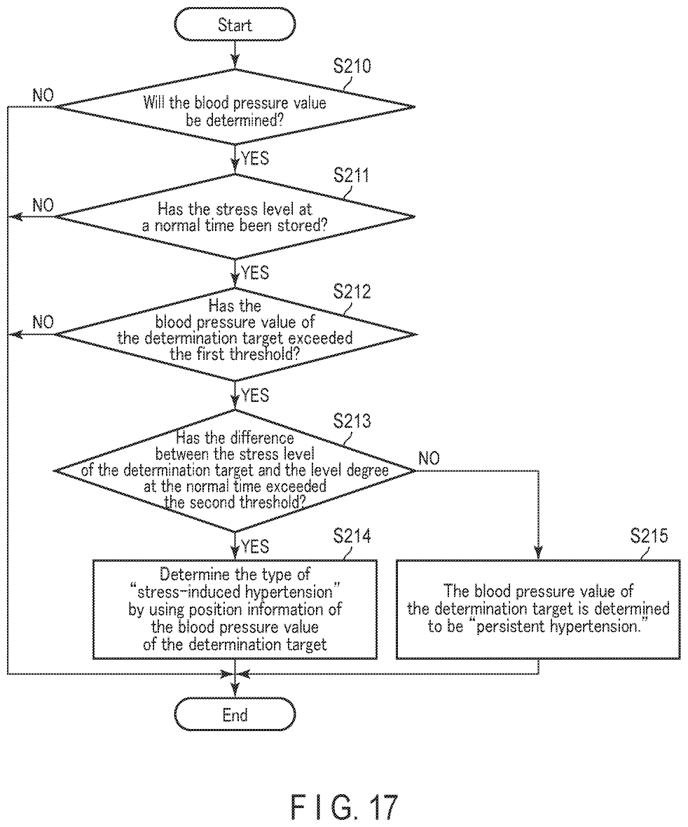

Next, an example of the blood pressure type determination operation of the information processing system including the information processing apparatus according to the second embodiment will be described with reference to FIG. 17. FIG. 17 is a flowchart illustrating an example of a processing procedure of the information processing system. The processing procedure described below is merely an example, and each processing may be changed as appropriate. In the processing procedure described below, steps can be omitted, replaced, and added as appropriate according to the embodiment.

[Step S210] to [Step S213]

The operations of steps S210 to S213 are the same as the operations of steps S110 to S113 in FIG. 10.

[Step S214]

When the blood pressure type determination unit 58-1 determines that the difference between the stress level of the determination target and the stress level in the normal state has exceeded the second threshold (Yes in step S213), the blood pressure type determination unit 58-1 determines the location information associated with the stress level of the determination target and the blood pressure value of the determination target.

The blood pressure type determination unit 58-1 determines the type of blood pressure based on the location information. Specifically, when determining that the measurement location is “hospital” from the location information, the blood pressure type determination unit 58-1 determines that the blood pressure value of the determination target is “white coat hypertension” and outputs the determination result. When determining that the measurement location is “workplace” from the location information, the blood pressure type determination unit 58-1 determines that the blood pressure value of the determination target is “workplace hypertension” and outputs the determination result. The determination result may be stored in the memory 41b or the storage unit 42 of the server SV, or may be output to the mobile information terminal IT or the doctor terminal DT. Any method may be used to determine the location from the location information.

[Step S215]

The operation of step S215 is the same as the operation of step S115 in FIG. 10.

<2-3> Effects

According to the above-described embodiment, the information processing system can determine the type of blood pressure in more detail by further considering the location information in the blood pressure type determination operation described in the first embodiment.

### <3> Third Embodiment

A third embodiment will be described. The third embodiment is different from the first embodiment in that the calculation timing of the stress level is different. The basic configuration and basic operation of the information processing system including the information processing apparatus according to the third embodiment are the same as those of the information processing system including the information processing apparatus according to the first embodiment described above. Therefore, descriptions of the matters described in the first embodiment and matters that can be easily analogized from the first embodiment will be omitted.

[3-1] Configuration

<3-1-1> Functional Configuration of Server

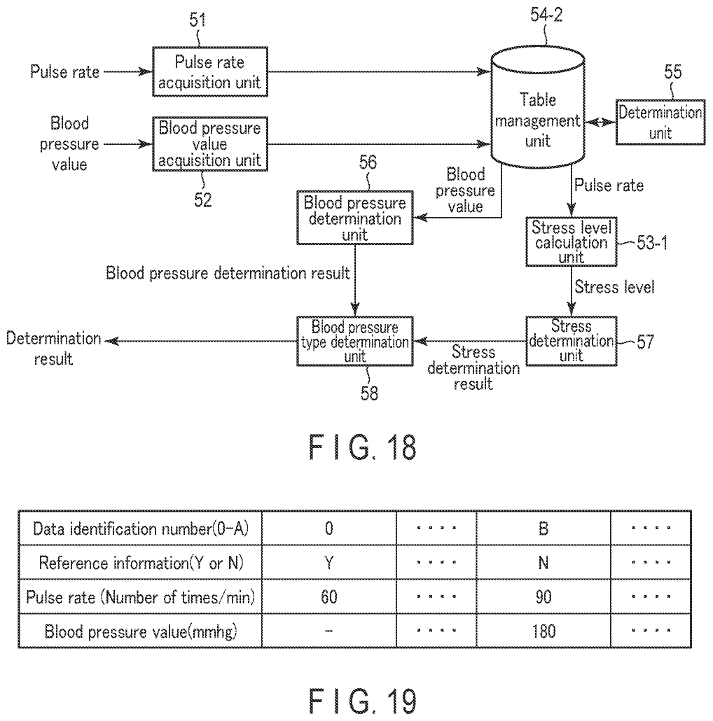

Next, an example of a functional configuration of the server SV according to the present embodiment will be described with reference to FIG. 18. FIG. 18 is a block diagram schematically illustrating an example of a functional configuration of the server SV according to the present embodiment. This embodiment is different from the first embodiment in that the table stores the pulse rate instead of the stress level. The third embodiment is different from the first embodiment in the timing of calculating the stress level.

The controller 41 of the server SV loads the program stored in the storage unit 42 into the memory 41b. Then, the controller 41 causes the processor 41a to interpret and execute the program loaded in the memory 41b and controls each component. Accordingly, the server SV according to the present embodiment functions as a computer including the pulse rate acquisition unit 51, the blood pressure value acquisition unit 52, a stress level calculation unit 53-1, a table management unit 54-2, the determination unit 55, the blood pressure determination unit 56, the stress determination unit 57, and the blood pressure type determination unit 58.

The table management unit 54-2 includes a table for each user. The table is developed in, for example, the memory 41b or the storage unit 42 of the server SV. The table stores blood pressure values and pulse rates received via the network NW. A specific structure example of the table will be described later. The table management unit 54-2 can display the information on the mobile information terminal IT or the doctor terminal DT in response to instructions from the user via the mobile information terminal IT or the doctor terminal DT.

The stress level calculation unit 53-1 calculates the stress level based on the pulse rate received via the table management unit 54-2.

<3-1-2> Structure Example of Table

Next, an example of the structure of a table will be described with reference to FIG. 19. FIG. 19 is a diagram illustrating an example of a structure of a table. For simplicity, the structure of the table will be described focusing on one user.

As illustrated in FIG. 19, the table stores, for example, a data identification number, reference information, a pulse rate, and a blood pressure value for each user ID included in the measurement data.

It should be noted that the blood pressure information may not be stored in a column related to the pulse rate at a normal time (the pulse rate when the reference information is Y).

<3-2> Operation

<3-2-1> Measurement Data Storage Operation

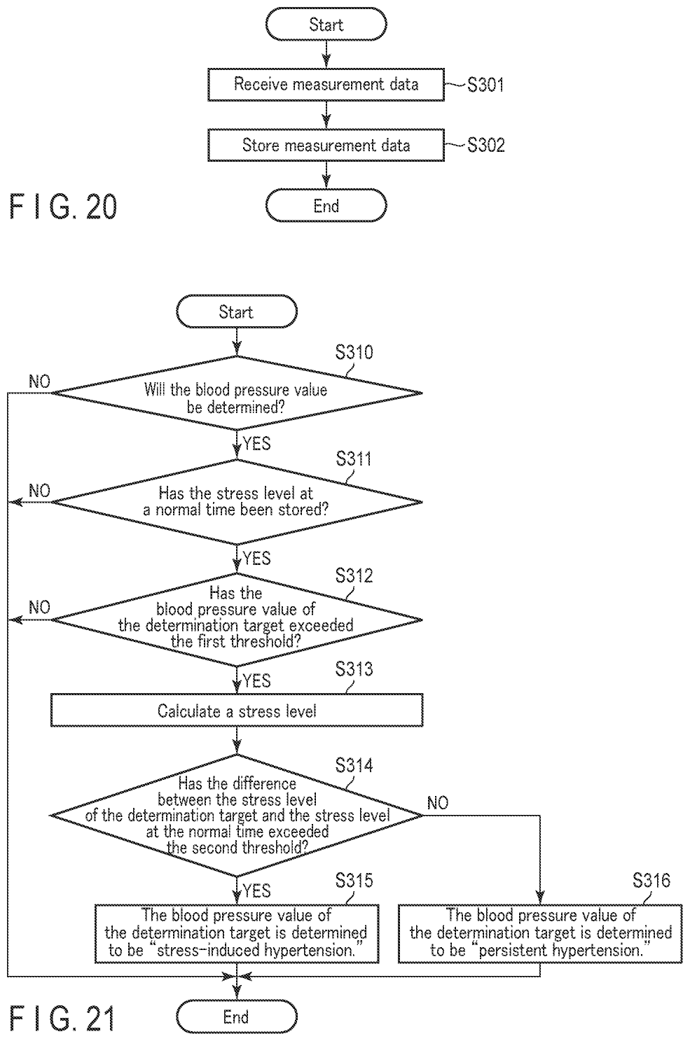

Next, an example of a measurement data storage operation of the information processing system including the information processing apparatus according to the third embodiment will be described with reference to FIG. 20. FIG. 20 is a flowchart illustrating an example of a processing procedure of the information processing system. The processing procedure described below is merely an example, and each processing may be changed as appropriate. In the processing procedure described below, steps can be omitted, replaced, and added as appropriate according to the embodiment.

[Step S301]

The operation in step S301 is the same as the operation in step S101 in FIG. 9.

[Step S302]

The table management unit 54-2 stores the reference information, the pulse rate, and the blood pressure value in the table based on the user ID.

<3-2-2> Blood Pressure Type Determination Operation

Next, an example of the blood pressure type determination operation of the information processing system including the information processing apparatus according to the third embodiment will be described with reference to FIG. 21. FIG. 21 is a flowchart illustrating an example of a processing procedure of the information processing system. The processing procedure described below is merely an example, and each processing may be changed as appropriate. In the processing procedure described below, steps can be omitted, replaced, and added as appropriate according to the embodiment.

[Step S310] to [Step S312]

The operations of steps S310 to S312 are the same as the operations of steps S110 to S112 in FIG. 10.

[Step S313]

When it is determined by the blood pressure determination unit 56 that the blood pressure value difference of the determination target has exceeded the first threshold (Yes in step S312), the stress level calculation unit 53-1 calculates the stress level based on the pulse rate supplied from the table management unit 54-2.

[Step S314] to [Step S316]

The operations in steps S314 to S316 are the same as the operations in steps S113 to S115 in FIG. 10.

<3-3> Advantageous Effects

According to the above-described embodiment, the information processing system can obtain the same effects as those described in the first embodiment even when the calculation timing of the stress level is changed.

### <4> Fourth Embodiment

A fourth embodiment will be described. In the fourth embodiment, a case where the second embodiment and the third embodiment are combined will be described. The basic configuration and basic operation of the information processing system including the information processing apparatus according to the fourth embodiment are the same as those of the information processing system including the information processing apparatus according to the first to third embodiments described above. Therefore, descriptions of matters described in the first to third embodiments and matters that can be easily analogized from the first to third embodiments will be omitted.

<4-1> Configuration

<4-1-1> Functional Configuration of Server

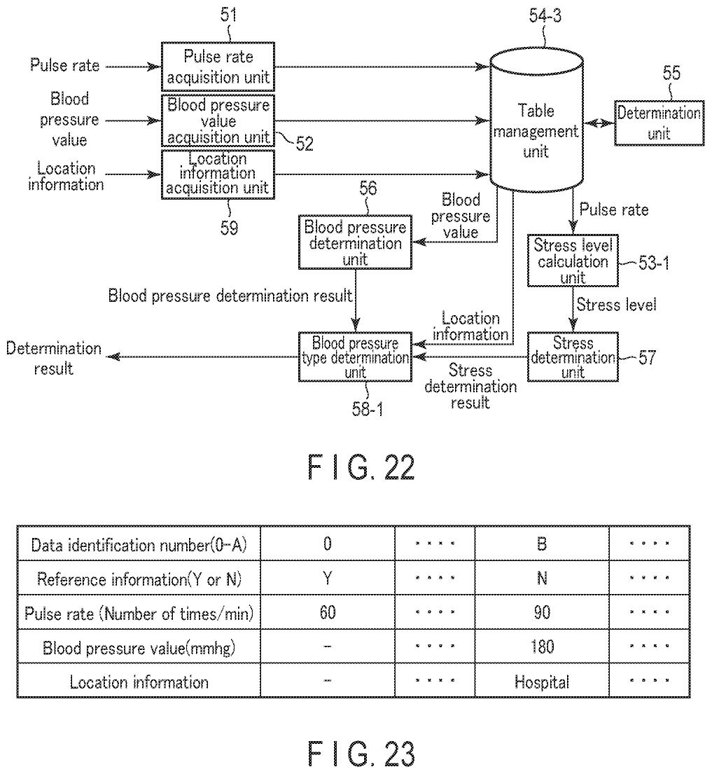

Next, an example of a functional configuration of the server SV according to the present embodiment will be described with reference to FIG. 22. FIG. 22 is a block diagram schematically illustrating an example of a functional configuration of the server SV according to the present embodiment.

The controller 41 of the server SV loads a program stored in the storage unit 42 into the memory 41b. Then, the controller 41 causes the processor 41a to interpret and execute the program loaded in the memory 41b and controls each component. Accordingly, the server SV according to the present embodiment functions as a computer including the pulse rate acquisition unit 51, the blood pressure value acquisition unit 52, the stress level calculation unit 53-1, a table management unit 54-3, the determination unit 55, the blood pressure determination unit 56, the stress determination unit 57, the blood pressure type determination unit 58-1, and the location information acquisition unit 59.

The table management unit 54-3 includes a table for each user. The table is loaded in, for example, the memory 41b or the storage unit 42 of the server SV. The table stores blood pressure values, pulse rates, and location information received via the network NW. A specific structure example of the table will be described later. The table management unit 54-3 can display the information on the mobile information terminal IT or the doctor terminal DT in response to instructions from the user via the mobile information terminal IT or the doctor terminal DT.

<4-1-2> Table Structure Example

Next, an example of the structure of the table will be described with reference to FIG. 23. FIG. 23 is a diagram illustrating an example of a structure of a table. For simplicity, the structure of the table will be described focusing on one user.

As illustrated in FIG. 23, the table stores, for example, a data identification number, reference information, a pulse rate, a blood pressure value, and location information for each user ID included in the measurement data.

The blood pressure information and the location information may not be stored in a column related to the pulse rate at the normal time (the pulse rate when the reference information is Y).

<4-2> Operation

<4-2-1> Measurement Data Storage Operation

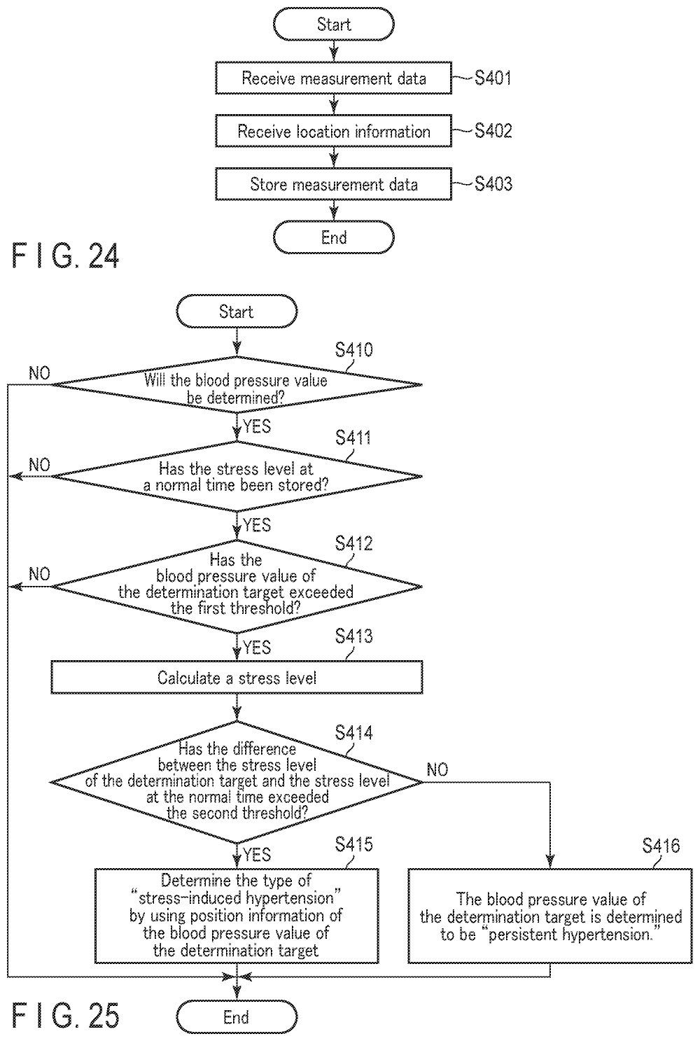

Next, an example of a measurement data storage operation of the information processing system including the information processing apparatus according to the fourth embodiment will be described with reference to FIG. 24. FIG. 24 is a flowchart illustrating an example of a processing procedure of the information processing system. The processing procedure described below is merely an example, and each processing may be changed as appropriate. In the processing procedure described below, steps can be omitted, replaced, and added as appropriate according to the embodiment.

[Step S401] and [Step S402]

The operations in steps S401 and S402 are the same as the operations in steps S201 and S202 in FIG. 16.

[Step S403]

The table management unit 54-3 stores the reference information, the pulse rate, the blood pressure value, and the location information in the table based on the user ID.

<4-2-2> Blood Pressure Type Determination Operation

Next, an example of the blood pressure type determination operation of the information processing system including the information processing apparatus according to the fourth embodiment will be described with reference to FIG. 25. FIG. 25 is a flowchart illustrating an example of a processing procedure of the information processing system. The processing procedure described below is merely an example, and each processing may be changed as appropriate. In the processing procedure described below, steps can be omitted, replaced, and added as appropriate according to the embodiment.

[Step S410] to [Step S412]

The operations of steps S410 to S412 are the same as the operations of steps S110 to S112 in FIG. 10.

[Step S413]

The operation of step S413 is the same as the operation in step S313 in FIG. 21.

[Step S414]

The operation of step S414 is the same as the operation of step S113 in FIG. 10.

[Step S415]

The operation in step S415 is the same as the operation in step S214 in FIG. 17.

[Step S416]

The operation of step S416 is the same as the operation of step S115 in FIG. 10.

<4-3> Advantageous Effects

According to the above-described embodiments, even when the second embodiment and the third embodiment are combined, the same effects as those described in the second embodiment can be obtained.

### <5> Fifth Embodiment

A fifth embodiment will be described. In the fifth embodiment, an example in which the pulse rate of the user is measured by the mobile information terminal will be described. The basic configuration and basic operation of the information processing system including the information processing apparatus according to the fifth embodiment are the same as those of the information processing system including the information processing apparatus according to the first to fourth embodiments described above. Therefore, descriptions of matters described in the first to fourth embodiments and matters that can be easily analogized from the first to fourth embodiments will be omitted.

In the first to fourth embodiments, the blood pressure monitor BT measures the pulse rate of the user (subject). However, the pulse may be measured by a terminal other than the blood pressure monitor BT. In the present embodiment, an example in which a mobile information terminal measures a pulse rate will be described.

<5-1> Mobile Information Terminal

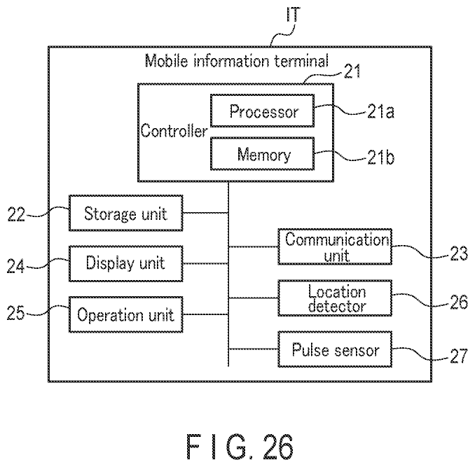

An example of a specific structure of the mobile information terminal IT will be described with reference to FIG. 26. FIG. 26 is a block diagram illustrating a configuration example of the mobile information terminal IT.

As illustrated in FIG. 26, the mobile information terminal IT includes a controller 21, a storage unit 22, a communication unit 23, a display unit 24, an operation unit 25, a location detector 26, a pulse sensor 27, and the like.

The pulse sensor 27 has, for example, the same configuration as the pulse sensor 17b of the blood pressure monitor BT.

<5-2> Method for Measuring Pulse Rate

Here, an example of a method of measuring the pulse rate will be described.

There are two major methods for measuring a pulse rate. The first is a method of measuring only a pulse rate, and the second is a method of simultaneously measuring a pulse rate and a blood pressure value.

As an example of a method of simultaneously measuring the pulse rate and the blood pressure value, a method of measuring the pulse rate by the pulse sensor 27 of the mobile information terminal IT while the blood pressure value is measured by the blood pressure monitor BT is considered.

Specifically, when a user (subject) starts measuring the pulse rate with the mobile information terminal IT, the controller 21 notifies the blood pressure monitor BT of the start of measuring the pulse rate via the communication unit 23.

Upon receiving the notification that the measurement of the pulse rate has been started, the controller 11 of the blood pressure monitor BT starts measuring the blood pressure value of the user.

When the measurement of the blood pressure value is completed, the controller 11 of the blood pressure monitor BT notifies the mobile information terminal IT of the completion of the measurement via the communication unit 12.

When the controller 21 of the mobile information terminal IT receives the information to the effect that the measurement of the blood pressure value is completed, the controller 21 ends the measurement of the pulse rate.

Then, the controller 21 of the mobile information terminal IT associates the pulse rate with the blood pressure value (measurement data) transmitted from the blood pressure monitor BT.

The above-described method is an example. Other methods are also applicable as long as it can be ensured that the measurement of the blood pressure value and the measurement of the pulse rate are performed substantially simultaneously.

<5-3> Advantageous Effects

According to the above-described embodiment, the information processing system measures the pulse rate and the blood pressure value using different terminals. Also in this case, the same effects as those of the first to fourth embodiments described above can be obtained.

<6> Modification

In the embodiments described above, the server SV has been described as an example of the “information processing equipment IPE” of the application example. The “information processing equipment IPE” of the application example may be realized by a plurality of servers SV. The “information processing equipment IPE” of the application example may be a mobile information terminal IT, a doctor terminal DT, or the like. For example, a case where the “information processing equipment IPE” of the application example is realized by the mobile information terminal IT will be briefly described. The controller 21 of the mobile information terminal IT loads the program stored in the storage unit 22 into the memory 21b. Then, the controller 21 causes the processor 21a to interpret and execute the program loaded in the memory 21b, thereby realizing the above-described functional configuration. In addition, a case where the “information processing equipment IPE” of the application example is realized by the doctor terminal DT will be briefly described. A controller 31 of the doctor terminal DT loads a program stored in a storage unit 32 into a memory 31b. Then, the controller 31 causes a processor 31a to interpret and execute the program loaded in the memory 31b, thereby realizing the above-described functional configuration.

The method of associating the reference information with the measurement data (pulse rate) has been described in the first embodiment, but the present invention is not limited thereto. For example, the reference information may be derived based on the location information included in the measurement data. As a specific example, for example, the table management unit 54-1 sets the reference information to “Y” in a case where it is determined from the received location information that the subject is at a location where the subject stays in a normal state. In addition, the table management unit 54-1 sets the reference information to “N” in a case where it is determined from the received location information that the subject is at a location where the subject does not normally stay. The operation subject may be another terminal. For example, a blood pressure monitor, a mobile information terminal, a doctor terminal, or the like may be used.

The present invention is not limited to the above-described embodiment as it is and can be embodied by modifying the constituent elements without departing from the scope thereof at the implementation stage. Further, various inventions can be formed by appropriately combining a plurality of components disclosed in the above embodiments. For example, some components may be deleted from all the components shown in the embodiments. Furthermore, the components of different embodiments may be combined as appropriate.

Some or all of the above embodiments may be described as in the following supplementary notes but are not limited thereto.

(Supplementary Note 1)

An information processing apparatus comprising:

a blood pressure value acquisition unit configured to acquire a blood pressure value of a subject measured by a blood pressure measurement unit;

a pulse rate acquisition unit configured to acquire a first pulse rate of the subject at a normal time and a second pulse rate of the subject in a time period in which the blood pressure value is measured;

a calculation unit configured to calculate a first tension degree of an autonomic nerve of the subject based on the first pulse rate and to calculate a second tension degree of the autonomic nerve of the subject based on the second pulse rate; and

a determination unit configured to determine a type of blood pressure of the blood pressure value based on the blood pressure value, the first tension degree, and the second tension degree.

(Supplementary Note 2)

An information processing method performed by an apparatus that processes a blood pressure value measured by a blood pressure measurement unit, the method comprising:

acquiring a blood pressure value of a subject measured by the blood pressure value measurement unit;

acquiring a first pulse rate of the subject at a normal time and a second pulse rate of the subject in a time period in which the blood pressure value is measured;

calculating a first tension degree of an autonomic nerve of the subject based on the first pulse rate and calculating a second tension degree of the autonomic nerve of the subject based on the second pulse rate; and

determining a type of blood pressure of the blood pressure value based on the blood pressure value, the first tension degree, and the second tension degree.

### REFERENCE SIGNS LIST

11, 21, 31, 41 Controller

11a, 21a, 31a, 41a Processor

11b, 21b, 31b, 41b Memory

12, 23, 33, 43 Communication unit

13, 22, 32, 42 Storage unit

14, 25, 35 Operation unit

15, 24, 34 Display unit

16 Acceleration sensor

17 Vital sensor

17a Blood pressure sensor

17b, 27 Pulse sensor

18 Environmental sensor

18a Temperature sensor

26 Location detector

51 Pulse rate acquisition unit

52 Blood pressure value acquisition unit

53, 53-1 stress level calculation unit

54, 54-1, 54-2, 54-3 Table management unit

55 Determination unit

56 Blood pressure determination unit

57 Stress determination unit

58, 58-1 Blood pressure type determination unit

59 Location information acquisition unit

BT1-BTn Blood pressure monitor

DT1-DTm Doctor terminal

IT1-Itn Mobile information terminal

UT1-Utn User terminal

1. An information processing apparatus comprising:
a processor configured to:
acquire a blood pressure value of a subject measured by a blood pressure measurement unit;
acquire a first pulse rate of the subject at a normal time and a second pulse rate of the subject in a time period in which the blood pressure value is measured;
calculate a first tension degree of an autonomic nerve of the subject based on the first pulse rate;
calculate a second tension degree of the autonomic nerve of the subject based on the second pulse rate;
determine whether or not the blood pressure value is classified as hypertension based on the blood pressure value; and
determine a type of blood pressure of the blood pressure value based on a comparison between the first tension degree and the second tension degree only when it is determined that the blood pressure value is classified as hypertension.

2. The information processing apparatus according to claim 1, wherein the processor is further configured to:
determine whether or not the subject is in a stressed state at the time of measuring the blood pressure value, based on the comparison between the first tension degree and the second tension degree; and
determine that the blood pressure value is suspected of being stress-induced hypertension when it is determined that the subject is in the stressed state at the time of measuring the blood pressure value.

3. The information processing apparatus according to claim 2, wherein the processor is further configured to determine that the blood pressure value is suspected of being persistent hypertension when it is determined that the subject is not in the stressed state at the time of measuring the blood pressure value.

4. The information processing apparatus according to claim 2, the processor is further configured to:
acquire location information indicating a location at which the blood pressure value is measured; and
determine whether the type of stress-induced hypertension is a white coat hypertension, a workplace hypertension, or hypertension associated with any other location, based on the location information when it is determined that the blood pressure value is suspected of being stress-induced hypertension.

5. The information processing apparatus according to claim 2, wherein the processor is further configured to output information indicating a determination result.

6. The information processing apparatus according to claim 2, wherein the processor is further configured to output information recommending measurement of a blood pressure at a normal time.

7. An information processing method performed by an apparatus that processes a blood pressure value measured by a blood pressure measurement unit, the method comprising:
acquiring a blood pressure value of a subject measured by the blood pressure value measurement unit;
acquiring a first pulse rate of the subject at a normal time and a second pulse rate of the subject in a time period in which the blood pressure value is measured;
calculating a first tension degree of an autonomic nerve of the subject based on the first pulse rate;
calculating a second tension degree of the autonomic nerve of the subject based on the second pulse rate;
determining whether or not the blood pressure value is classified as hypertension based on the blood pressure value; and
determining a type of blood pressure of the blood pressure value based on a comparison between the first tension degree and the second tension degree only when it is determined that the blood pressure value is classified as hypertension.

8. A non-transitory computer readable medium including computer executable instructions, wherein the instructions, when executed by a processor, cause the processor to perform a method comprising:
acquiring a blood pressure value of a subject measured by a blood pressure value measurement unit;
acquiring a first pulse rate of the subject at a normal time and a second pulse rate of the subject in a time period in which the blood pressure value is measured;
calculating a first tension degree of an autonomic nerve of the subject based on the first pulse rate;
calculating a second tension degree of the autonomic nerve of the subject based on the second pulse rate;
determining whether or not the blood pressure value is classified as hypertension based on the blood pressure value; and
determining a type of blood pressure of the blood pressure value based on a comparison between the first tension degree and the second tension degree only when it is determined that the blood pressure value is classified as hypertension.
